(19) United States
(12) Patent Application Publication
(10) Pub. No.: US 2025/0025027 A1
(43) Pub. Date: Jan. 23, 2025

(54) IMAGE PICKUP UNIT AND ENDOSCOPE

(71) Applicant: OLYMPUS MEDICAL SYSTEMS CORP., Tokyo (JP)

(72) Inventor: Shota NAKAO, Tokyo (JP)

(73) Assignee: OLYMPUS MEDICAL SYSTEMS CORP., Tokyo (JP)

(21) Appl. No.: 18/906,558

(22) Filed: Oct. 4, 2024

Related U.S. Application Data
(63) Continuation of application No. PCT/JP2022/029668, filed on Aug. 2, 2022.

(51) Int. Cl. A61B 1/05

(52) U.S. Cl. CPC A61B 1/05

(57) ABSTRACT

In an image pickup unit, an image pickup device, a first substrate, and a second substrate are connected in the order from a distal end. The image pickup unit includes a cutout that is provided in a side surface of the first substrate, penetrates from a distal end surface to a proximal end surface, and has a smaller width dimension of an opening on the proximal end surface side than that of an opening of the distal end surface side, and a resin filled in the cutout, a gap between the image pickup device and the first substrate, and a gap between the first substrate and the second substrate.

### CROSS-REFERENCE TO RELATED APPLICATION

This application is a continuation application of PCT/JP2022/029668 filed on Aug. 2, 2022, the entire contents of which are incorporated herein by this reference.

### BACKGROUND OF THE INVENTION

### 1. Field of the Invention

The present disclosure relates to an image pickup unit in which an image sensor and a plurality of circuit substrates are stacked and packaged, and an endoscope including the image pickup unit.

### 2. Description of the Related Art

Conventionally, endoscopes have been widely used in medical fields and industrial fields for various inspection. The endoscope includes an insertion portion configured to be inserted into a subject.

The insertion portion of the endoscope is provided with an image pickup unit including an image sensor and a circuit substrate on which an electronic component or the like that drives the image sensor on a distal end. In such an image pickup unit, an underfill agent including an epoxy resin as a main agent is filled in a gap between the image sensor and the circuit substrate, for example.

For example, WO 2016/203797 describes that a cutout portion for applying an underfill agent is formed in a circuit substrate. The cutout portion is used to insert a dispense nozzle when injecting the underfill agent in a gap between an image pickup device and a circuit substrate.

The underfill agent is filled in a gap between an image sensor and the circuit substrate of an image pickup unit.

### SUMMARY OF THE INVENTION

An image pickup unit according to one aspect of the present disclosure includes an image pickup device, a first substrate, and a second substrate, and the image pickup device, the first substrate, and the second substrate are connected in the order from a distal end of the image pickup unit. The image pickup unit further includes a cutout that is provided on a side surface of the first substrate and penetrates from a distal end surface to a proximal end surface, the cutout being formed such that a width dimension of a second opening on a side of the proximal end surface is smaller than a width dimension of a first opening on a side of the distal end surface in a direction along the side surface, and a resin filled in the cutout, a gap between the image pickup device and the first substrate, and a gap between the first substrate and the second substrate.

An image pickup unit according to another aspect of the disclosure includes an image pickup device, a first substrate, and a second substrate, and the image pickup device, the first substrate, and the second substrate are connected in the order from a distal end of the image pickup unit. The image pickup unit further includes a cutout that is provided on a side surface of the first substrate and penetrates from a distal end surface to a proximal end surface, the cutout being formed such that a width dimension of a second opening on a side of the proximal end surface is larger than a width dimension of a first opening on a side of the distal end surface in a direction along the side surface, and a resin filled in the cutout, a gap between the image pickup device and the first substrate, and a gap between the first substrate and the second substrate.

An endoscope according to one aspect of the disclosure includes an insertion portion configured to be inserted in a subject, and an image pickup unit that is provided in the insertion portion, and includes an image pickup device, a first substrate, and a second substrate, the image pickup device, the first substrate, and the second substrate being connected in the order from a distal end of the image pickup unit. The image pickup unit further includes a cutout that is provided on a side surface of the first substrate and penetrates from a distal end surface to a proximal end surface, the cutout being formed such that a width dimension of a second opening on a side of the proximal end surface is smaller than a width dimension of a first opening on a side of the distal end surface in a direction along the side surface, and a resin filled in the cutout, a gap between the image pickup device and the first substrate, and a gap between the first substrate and the second substrate.

### BRIEF DESCRIPTION OF THE DRAWINGS

FIG. 1 is a view showing a configuration of an endoscope apparatus.

FIG. 2 is a perspective view showing a configuration of an image pickup unit.

FIG. 3 is an exploded perspective view showing the configuration of the image pickup unit.

FIG. 4 is a side view showing the configuration of the image pickup unit.

FIG. 5 is a plan view showing a configuration of a planar circuit substrate.

FIG. 6 is a rear view showing the configuration of the planar circuit substrate.

FIG. 7 is a side view showing the configuration of the image pickup unit in a state where an underfill agent is applied to a gap between an image pickup device and a planar circuit substrate by a dispense nozzle.

FIG. 8 is a side view showing the configuration of the image pickup unit in a state where the underfill agent is filled also in a recessed portion of an odd-shaped circuit substrate by the dispense nozzle.

FIG. 9 is a side view showing a configuration of an image pickup unit of a first modification.

FIG. 10 is a side view showing a configuration of an image pickup unit of a second modification.

FIG. 11 is a side view showing a configuration of an image pickup unit of a third modification.

FIG. 12 is a plan view showing a configuration of a planar circuit substrate of a fourth modification.

FIG. 13 is a rear view showing the configuration of the planar circuit substrate of the fourth modification.

FIG. 14 is a plan view showing a configuration of a planar circuit substrate of a fifth modification.

FIG. 15 is a rear view showing the configuration of the planar circuit substrate of the fifth modification.

FIG. 16 is a side view showing a configuration of an image pickup unit of a sixth modification.

FIG. 17 is a side view showing the configuration of the image pickup unit in a state where the underfill agent is filled in a recessed portion of an odd-shaped circuit substrate by the dispense nozzle of the sixth modification.

FIG. 18 is a side view showing the configuration of the image pickup unit in a state where the underfill agent is applied also to a gap between the image pickup device and the planar circuit substrate by the dispense nozzle of the sixth modification.

### DETAILED DESCRIPTION OF THE PREFERRED EMBODIMENT(S)

An image pickup unit and an endoscope as an insertion device in which the image pickup unit is provided on a distal end of an insertion portion of the present disclosure will be described below as an example. Note that in the description below, the drawings based on each embodiment is schematic, and care should be taken to the fact that the relation between the thickness and width of each portion, the ratio in thickness of each portions, and the like are different from the actual ones, and there is a case where the respective drawings include some portions in which the relation and ratios among the dimensions are different from each other.

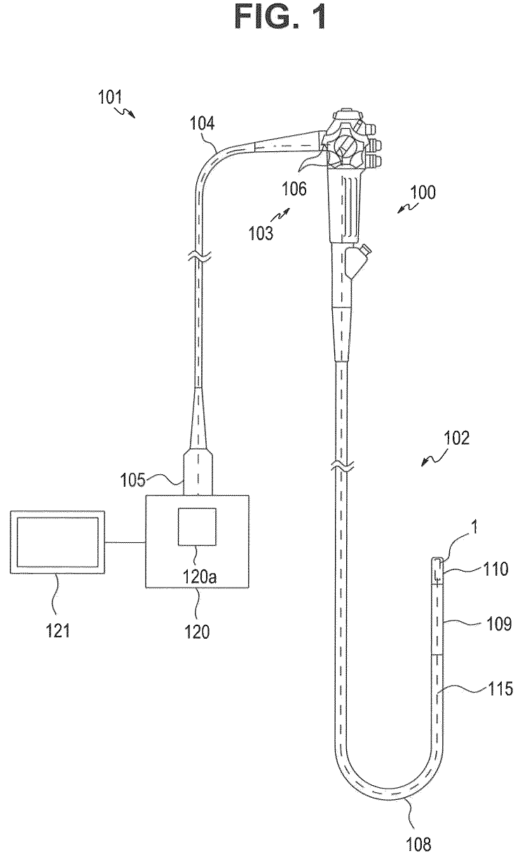

First, one example of a configuration of an endoscope apparatus 101 including an image pickup unit 1 of one aspect of the present disclosure will be described with reference to FIG. 1. The endoscope apparatus 101 as an endoscope system of the present embodiment includes an endoscope 100, an external apparatus 120, and an image display unit 121.

The endoscope 100 is configured to be insertable into a subject such as a human body, and to optically pickup an image of a predetermined observation site in the subject. Note that the subject into which the endoscope 100 is inserted is not limited to a human body, but may be another living body, or an artificial object such as a machine or a structure.

The endoscope 100 includes an insertion portion 102, an operation portion 103, and a universal cord 104. The insertion portion 102 is introduced (inserted) inside of the subject. The operation portion 103 is continuously provided at a proximal end of the insertion portion 102. The universal cord 104 is extended from a side portion of the operation portion 103.

The insertion portion 102 includes a distal end portion 110, a bending portion 109, and a flexible tube portion 108.

The distal end portion 110 is disposed on a distal end. The bending portion 109 is disposed on a proximal end side of the distal end portion 110. The bending portion 109 includes a bendable structure. The flexible tube portion 108 is flexible. The flexible tube portion 108 is continuously provided on a proximal end side of the bending portion 109. The flexible tube portion 108 is then connected to a distal end side of the operation portion 103.

The endoscope 100 here is exemplified, for example, in the form of a so-called flexible endoscope with the flexible insertion portion 102. Note that the endoscope 100 may be a so-called rigid endoscope with the rigid insertion portion 102.

The image pickup unit 1 is provided on the distal end portion 110. The image pickup unit 1 here is an image pickup module of a CSP (chip size package).

The operation portion 103 is provided with an angle operation knob 106. The angle operation knob 106 is an operation lever for operating bending of the bending portion 109.

An endoscope connector 105 is provided at a proximal end portion of the universal cord 104. The endoscope connector 105 is detachably connected to the external apparatus 120. The external apparatus 120 is connected to the image display unit 121 such as a monitor, through a cable.

In addition, the endoscope 100 includes a composite cable 115 inserted through the universal cord 104, the operation portion 103, and the insertion portion 102. Various electric cables, optical fiber bundles (not shown) or the like are inserted through the composite cable 115. The optical fiber bundle transmits an illumination light from a light source unit provided in the external apparatus 120.

The various electric cables in the composite cable 115 are configured to electrically connect the endoscope connector 105 and the image pickup unit 1. The endoscope connector 105 is connected to the external apparatus 120. This allows the image pickup unit 1 to be electrically connected to the external apparatus 120 through the composite cable 115.

Through the composite cable 115, electric power is supplied from the external apparatus 120 to the image pickup unit 1, and the external apparatus 120 and the image pickup unit 1 communicate with each other.

The external apparatus 120 is provided with an image processing unit 120a. The image processing unit 120a generates a video signal based on an image pickup device output signal (image signal) outputted from the image pickup unit 1. The image processing unit 120a then outputs the video signal to the image display unit 121. Specifically, in the present embodiment, an optical image (endoscopic image) obtained by image pickup with the image pickup unit 1 is displayed as a video on the image display unit 121.

The image display unit 121 is a display using liquid crystal or organic EL (electro luminescence), for example. Note that the endoscope 100 is not limited to the configuration connected to the external apparatus 120 or the image display unit 121, but may include a part or an entirety of the image processing unit and a monitor, for example.

In addition, the optical fiber bundle is configured to transmit a light emitted from the light source unit of the external apparatus 120 to an illumination window of the distal end portion 110. The illumination window functions as an illumination light emission unit. Furthermore, the light source unit is not limited to be disposed in the external apparatus 120, but may be disposed in the operation portion 103 or the distal end portion

110 of the endoscope 100. The light source unit may be configured using an LED or the like.

Next, description will be made on a configuration of the image pickup unit 1 provided in the distal end portion 110. Note that when up, down, left or right is indicated in the following description, unless otherwise stated, it would mean left or right when seen from the position opposite a front surface area of the image pickup unit 1.

Note that separately from the up, down, left, or right direction of the image pickup unit 1, it may also be described as an up and down direction towards a paper of the drawing, or a vertical up and down direction.

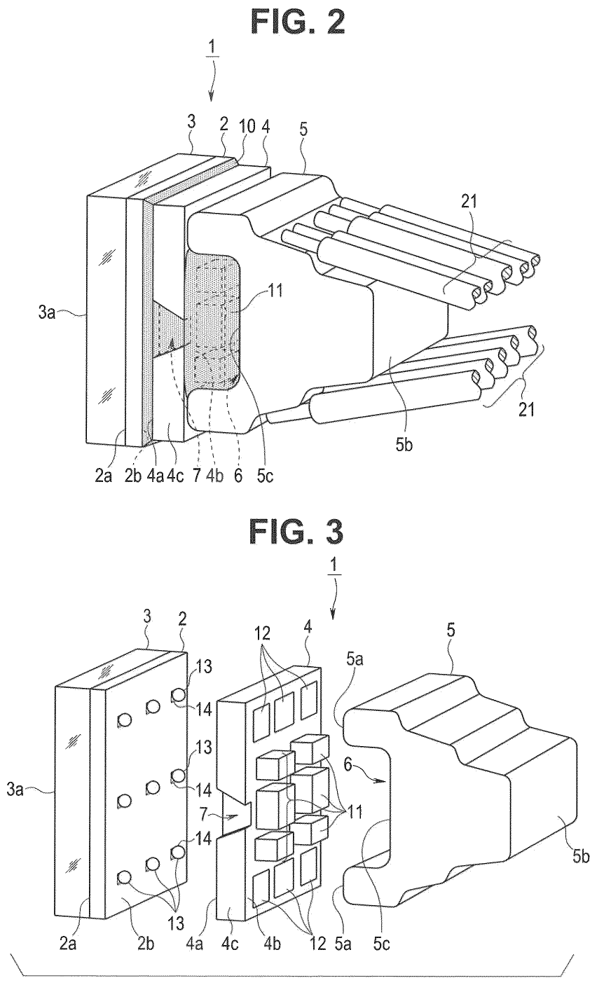

In the image pickup unit 1 of the present embodiment as shown in FIG. 2 and FIG. 3, a glass lid 3, an image pickup device 2, a planar circuit substrate 4 as a first circuit substrate (first substrate), and an odd-shaped circuit substrate 5 as a second circuit substrate (second substrate) are stacked in the order from the distal end.

The image pickup device 2 is an image sensor configured to receive a light of an optical image of an observation target formed by an image-forming optical lens unit (not shown). The image pickup device 2 performs predetermined photoelectric conversion processing on the received optical image. Thereby the image pickup device 2 generates an image signal of the observation target.

Here, the image pickup device 2 is an image sensor in a common form such as a CCD (charge coupled device) or a CMOS (complementary metal oxide semiconductor), for example.

The glass lid 3 of a protective glass (cover glass) is provided on a side of a front surface (light-receiving surface) 2a that is a distal end surface of the image pickup device 2. Note that in a state where the image pickup unit 1 is disposed inside the distal end portion 110 of the endoscope 100, an image-forming optical lens unit (not shown) is disposed in front (a distal end side) of the image pickup device 2.

In this state, the image-forming optical lens unit is arranged so that an optical axis (not shown) passes through a center of the light-receiving surface of the image pickup device 2 and substantially coincides with an axis perpendicular to the light-receiving surface (central axis). The front surface 2a of the image pickup device 2 is then orthogonal to the optical axis of the image-forming optical lens unit.

With such a configuration, a light from the observation target condensed by the image-forming optical lens unit enters from a front surface 3a that is a distal end surface of the glass lid 3. The light then transmits the glass lid 3 and is formed on the light-receiving surface of the image pickup device 2.

A plurality of electrodes 14 and a plurality of solder pastes 13 that are formed in the form of bumps on the plurality of electrodes 14 are provided on a rear surface 2b that is a proximal end surface of the image pickup device 2.

The planar circuit substrate 4 is a circuit substrate in a flat plate shape. The planar circuit substrate 4 is a multilayer structure substrate formed by stacking a plurality of substrates, for example.

As a substrate stacked in the planar circuit substrate 4, a ceramic substrates, an epoxy glass substrate, a flexible substrate, a glass substrate, a silicone substrate, or the like are used, for example. The planar circuit substrate 4 is provided substantially parallel to the image pickup device 2.

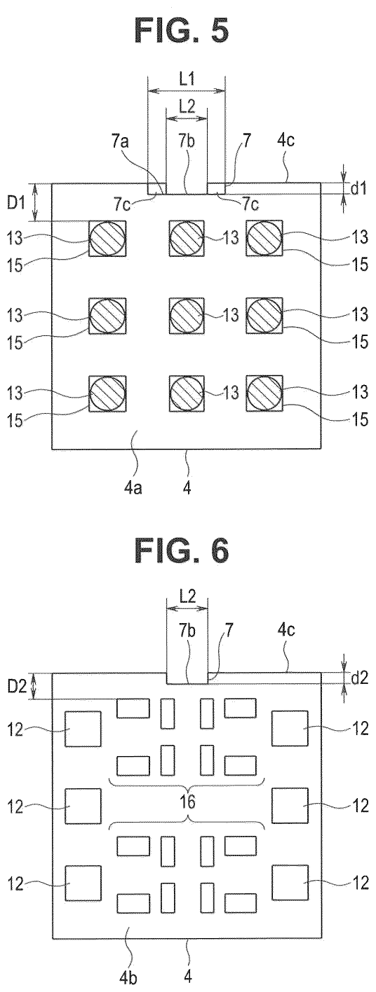

On a front surface 4a that is a distal end surface of the planar circuit substrate 4, connection lands 15 (not shown here, see FIG. 5) are provided. The connection land 15 are a plurality of electrodes electrically connected to the respective electrodes 14, to which the solder pastes 13 are applied, of the image pickup device 2. The connection lands 15 are electrically connected to the respective electrodes 14 of the image pickup device 2 by fusing the respective solder pastes 13 through a reflow process. With this, the planar circuit substrate 4 and the image pickup device 2 are electrically and mechanically connected to each other.

On a rear surface 4b that is a proximal end surface of the planar circuit substrate 4, as shown in FIG. 3, a plurality of electronic components 11 are mounted. The electronic components 11 are capacitors, resistors, coils, transistors, diodes, drive ICs, or the like, for example.

Furthermore, on the rear surface 4b of the planar circuit substrate 4, a plurality of electrodes 12 that are connection lands are provided. Each of the electrodes 12 is arranged in an edge area in an up and down direction of the planar circuit substrate 4. The electrodes 12 are connection electrodes for connecting the planar circuit substrate 4 to the odd-shaped circuit substrate 5.

The odd-shaped circuit substrate 5 is a so-called three-dimensional circuit substrate including a three-dimensional structure. The odd-shaped circuit substrate 5 here is an MID (molded interconnect device) substrate in which a three-dimensional wiring is formed in an injection-molded resin component, for example. Note that the odd-shaped circuit substrate 5 is not limited to the MID substrate, but may be a ceramic substrate, an epoxy glass substrate, a glass substrate, or a silicone substrate, for example.

The odd-shaped circuit substrate 5 includes a front surface 5a. The front surface 5a is a connection surface for connecting the odd-shaped circuit substrate 5 to the rear surface 4b of the planar circuit substrate 4. Note that on the front surface 5a, a plurality of electrodes, not shown, electrically connected to the respective electrodes 12 of the planar circuit substrate 4 are provided at positions corresponding to the respective electrodes 12.

The front surface 5a of the odd-shaped circuit substrate 5 is vertically divided by a recessed portion 6 opened on a distal end side and side portion sides. The recessed portion 6 is formed at a substantially central portion in the up and down direction in front of the odd-shaped circuit substrate 5. Note that the electronic components 11 of the planar circuit substrate 4 are housed in the recessed portion 6 in a state where the odd-shaped circuit substrate 5 is connected to the planar circuit substrate 4.

With this, the odd-shaped circuit substrate 5 avoids interference with the electronic components 11 of the planar circuit substrate 4. Note that the odd-shaped circuit substrate 5 is formed with a plurality of cable connection lands (not shown) of the three-dimensional wiring on upper and lower surfaces. Respective image pickup cables 21 are connected to the plurality of cable connection lands by solder or the like. The plurality of image pickup cables 21 are bundled, and are inserted and arranged in the composite cable 115 shown in FIG. 1.

The planar circuit substrate 4 is formed with a cutout 7 having a recessed shape at a substantially center of one side surface 4c. The cutout 7 is formed in the one side surface 4c of the planar circuit substrate 4 over the front surface 4a and the rear surface 4b, with a trapezoidal surface parallel to the one side surface 4c. Note that the cutout 7 has a trapezoidal shape in which a first base 7a of an opening (first opening) on the front surface 4a side is longer than a second base 7b of an opening (second opening) of the rear surface 4b side (that is, the second base 7b is shorter than the first base 7a). Each of the openings of the cutout 7 is rectangular in shape here.

Furthermore, the cutout 7 has a predetermined depth. The cutout 7 includes two tapered walls 7c that are closer together (progressively smaller in dimension) from the front surface 4a to the rear surface 4b on each side.

The cutout 7 constitutes an injection portion into which a distal end of a dispense nozzle 130 is inserted when an underfill agent 10 which is a cured resin is filled in a gap (space) between the rear surface 2b of the image pickup device 2 and the front surface 4a of the planar circuit substrate 4.

Here, a dimensional relation among the image pickup device 2, the planar circuit substrate 4 in which the cutout 7 is formed, and the odd-shaped circuit substrate 5 in which the recessed portion 6 is formed, of the image pickup unit 1, will be described.

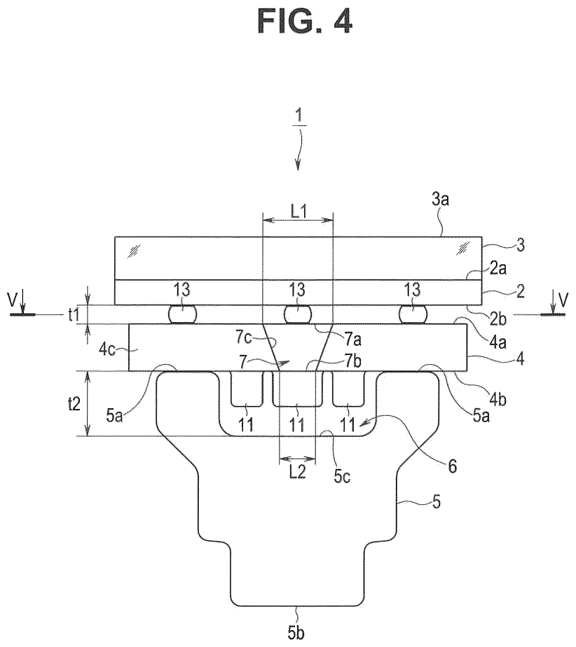

As shown in FIG. 4, the gap is formed between the image pickup device 2 and the planar circuit substrate 4. The gap has a distance t1. In other words, the image pickup unit 1 includes the gap of distance t1 between the rear surface 2b of the image pickup device 2 and the front surface 4a of the planar circuit substrate 4. That is, the distance t1 is a separation distance between the rear surface 2b which is the proximal end surface of the image pickup device 2 and the front surface 4a which is the distal end surface of the planar circuit substrate 4 as the first substrate.

A space is formed between the planar circuit substrate 4 and a bottom surface 5c of the recessed portion 6 of the odd-shaped circuit substrate 5. The space include a gap of a distance t2 in a height direction. In other words, the image pickup unit 1 includes the space of the distance t2 between the rear surface 4b which is the proximal end surface of the planar circuit substrate 4 as the first substrate and the bottom surface 5c of the recessed portion 6.

That is, the odd-shaped circuit substrate 5 includes the recessed portion 6 at the depth of the distance t2. The distance t2 is set to be longer than the distance t1 (t2>t1).

The cutout 7 formed in the one side surface 4c of the planar circuit substrate 4 includes a first base 7a of a length L1 on the front surface 4a side. That is, the length L1 is a dimension in a width direction (direction along a side of the one side surface 4c) of an opening (front surface opening) on the front surface 4a side (distal end side) of the cutout 7.

The cutout 7 includes a second base 7b of a length L2 on the rear surface 4b side. That is, the length L2 is a dimension in a width direction (direction along a side of the one side surface 4c) of an opening (rear surface opening) on the rear surface 4b side (proximal end side) of the cutout 7.

As above, in the cutout 7, the length L1 of the first base 7a of the opening on the distal end side is longer than the length L2 of the second base 7b of the opening on the proximal end side (L1>L2). That is, the cutout 7 is a trapezoidal shape in plan along the one side surface 4c of the planar circuit substrate 4. Such a cutout 7 is a recessed portion including a rectangular opening in each front and rear end surfaces (the front surface 4a and the rear surface 4b) and a trapezoidal opening in the one side surface 4c.

Note that the distance t1 of the gap between the image pickup device 2 and the planar circuit substrate 4 is about 100 μm, for example. Therefore, the length L1 of the first base 7a of the opening on the front surface 4a side of the cutout 7 is set to be longer than 100 μm (L1>t1) for example, and equal to or less than a maximum external shape of the planar circuit substrate 4.

Furthermore, the length L2 of the second base 7b of the opening on the rear surface 4b side of the cutout 7 not only needs to be shorter than the length L1 (L1>L2), but may preferably be longer than the distance t1 (L2>t1), considering the capillary action described below. Therefore, the length L2 is set to be longer than 100 μm (assumed distance t1) for example, and shorter than the length L1.

From the above description, respective dimensions of the image pickup unit 1 are set to the length L1>t1, L1>L2, L2>t1, and t2>t1. Note that the order of priority in each of dimensional relations is L1>t1 first, followed by L2>t1, L1>L2, and t2>t1.

Note that as shown in FIG. 5, the dimension of the depth d1 of the cutout 7 from the one side surface 4c on the front surface 4a side of the planar circuit substrate 4 is set to be equal to or less than a distance D1 (d1≤D1). Here, D1 is a distance from the one side surface 4c to the connection lands 15 (first connection lands) to each of which the solder paste 13 closest to the one side surface 4c is fused in a direction orthogonal to the one side surface 4c.

That is, the cutout 7 is formed to have the depth d1 of the opening on the front surface 4a side in the direction orthogonal to the one side surface 4c. On the front surface 4a of the planar circuit substrate 4, one ends of the connection lands 15 closest to the one side surface 4c side of the plurality of connection lands 15 are provided at positions of the distance D1 from the one side surface 4c.

The predetermined depth d1 of the opening of the cutout 7 on the front surface 4a side of the planar circuit substrate 4 is set to be equal to or less than the distance D1 (d1≤D1) from the one side surface 4c of the planar circuit substrate 4 to the connection lands 15.

As shown in FIG. 6, a dimension of a depth d2 of the cutout 7 from the one side surface 4c on the rear surface 4b side of the planar circuit substrate 4 is set to be equal to or less than a distance D2 (d2≤D2). Here, D2 is a distance from the one side surface 4c to SMD (surface mounting) connection lands 16 (second connection lands) on which the electronic components 11 closest to the one side surface 4c are mounted.

That is, the cutout 7 is formed to have the predetermined depth d2 of the opening on the rear surface 4b side in the direction orthogonal to the one side surface 4c. On the rear surface 4b of the planar circuit substrate 4, one ends of the SMD connection lands 16 closest to the one side surface 4c side of the plurality of SMD connection lands 16 are provided at positions of the distance D2 from the one side surface 4c.

The depth d2 of the opening of the cutout 7 on the rear surface 4b side of the planar circuit substrate 4 is set to be equal to or less than the distance D2 (d2≤D2) from the one side surface 4c of the planar circuit substrate 4 to the SMD connection lands 16.

Note that the dimensions of the depths d1 and d2 of the openings of the cutout 7 are considered to be sufficient so long as there is a recessed groove in which the underfill agent 10 can flow. Therefore, the dimensions of the depths d1 and d2 may be, for example, about 10 μm or more.

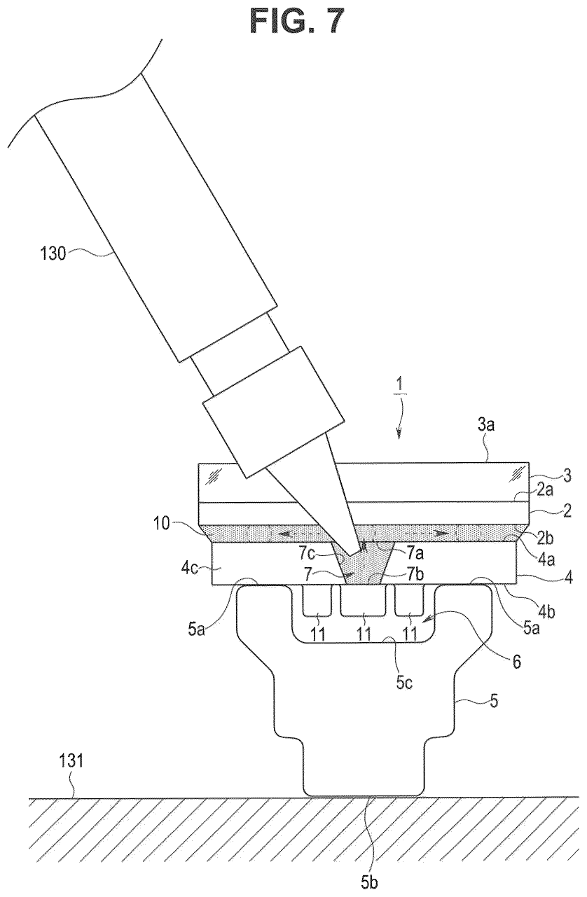

The image pickup unit 1 configured as above is placed with the glass lid 3 vertically above and the rear surface 5b of the odd-shaped circuit substrate 5 on an application stage 131 in an application process of the underfill agent 10, as shown in FIG. 7. In this state, a distal end of the dispense nozzle 130 is directed into the cutout 7 of the planar circuit substrate 4 of the image pickup unit 1. The underfill agent 10 is then applied from the cutout 7 of the image pickup unit 1.

At this time, the underfill agent 10 rises vertically upwards from the cutout 7 by the operation of the capillary action, and flows into a gap between the image pickup device 2 and the planar circuit substrate 4. Note that even if the distal end of the dispense nozzle 130 is directed to the odd-shaped circuit substrate 5 side on a vertically downward side, the underfill agent 10 blocked by the tapered walls 7c of the cutout 7 flows to the image pickup device 2 side with a larger opening on the vertical upward side of the cutout 7 by the capillary action.

That is, the length L1 of the first base 7a of the cutout 7, which is the width dimension of the opening (first opening) on the side of the gap between the image pickup device 2 and the planar circuit substrate 4, is set to be longer than the length L2 of the second base 7b of the cutout 7, which is the width dimension of the opening (second opening) on the side of a gap between the planar circuit substrate 4 and the odd-shaped circuit substrate 5 (L1>L2). And the distance t1 of the gap between the image pickup device 2 and the planar circuit substrate 4 is set to be shorter than the length L2 of the second base 7b (t1<L2). Thus, by the principle of capillary action, the underfill agent 10 is subjected to a large force to flow into the gap between the image pickup device 2 and the planar circuit substrate 4 on the vertically upward side against gravity.

In the image pickup unit 1, the distance t1 of the gap between the image pickup device 2 and the planar circuit substrate 4 is set to be shorter than the distance t2 of the gap between the planar circuit substrate 4 and the bottom surface 5c of the recessed portion 6 of the odd-shaped circuit substrate 5 (t1<t2). Thus, the underfill agent 10 is prevented from flowing into the recessed portion 6 side of the odd-shaped circuit substrate 5.

As above, the image pickup unit 1 is provided with the cutout 7 for applying the underfill agent 10 by the dispense nozzle 130 in the one side surface 4c of the planar circuit substrate 4. The cutout 7 has a structure where the opening on the image pickup device 2 side is wider than the opening on the odd-shaped circuit substrate 5.

The length L1 which is the width dimension of the opening on the image pickup device 2 side of the cutout 7 is set to be longer than the distance t1 of the gap between the image pickup device 2 and the planar circuit substrate 4 (L1>t1). Furthermore, in the cutout 7, the length L2 which is the width dimension of the opening on the odd-shaped circuit substrate 5 side is shorter than the length L1 which is the width dimension of the
opening of the image pickup device 2 side (L2<L1) to form a recessed shape including the tapered walls 7c on each side surfaces.

As above, the image pickup unit 1 is capable of applying the underfill agent 10 from the distal end of the dispense nozzle 130 by aiming a center portion of the cutout 7. The image pickup unit 1 is capable of applying the underfill agent 10 by the odd-shaped circuit substrate 5 on the proximal end side being placed on the application stage 131, without placing the glass lid 3 on the application stage 131.

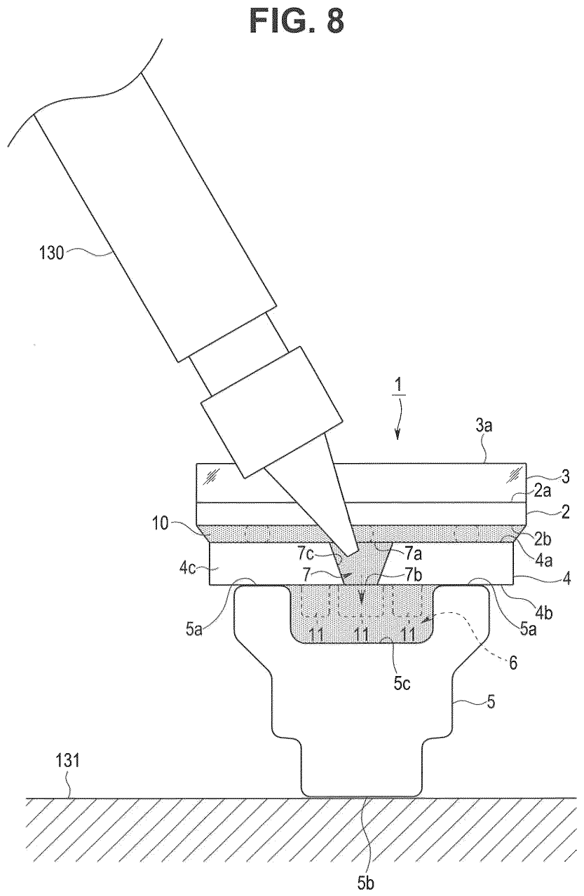

Note that the cutout 7 has a penetration shape including openings on both of the image pickup device 2 side and the odd-shaped circuit substrate 5 side in the one side surface 4c of the planar circuit substrate 4. Therefore, after filling the underfill agent 10 in the gap between the image pickup device 2 and the planar circuit substrate 4, the underfill agent 10 flows to the recessed portion 6 side, which is the gap between the planar circuit substrate 4 and the odd-shaped circuit substrate 5, as shown in FIG. 8. In the image pickup unit 1, the gap between the image pickup device 2 and the planar circuit substrate 4 and the gap (in the recessed portion 6) between the the planar circuit substrate 4 and the odd-shaped circuit substrate 5 can be filled with the underfill agent 10. Furthermore, in the image pickup unit 1, the underfill agent 10 applied from the cutout 7 can be filled while preventing from flowing outside. As a result, an external shape of the image pickup unit 1 can be prevented from becoming larger due to flowing out of the underfill agent 10.

### First Modification

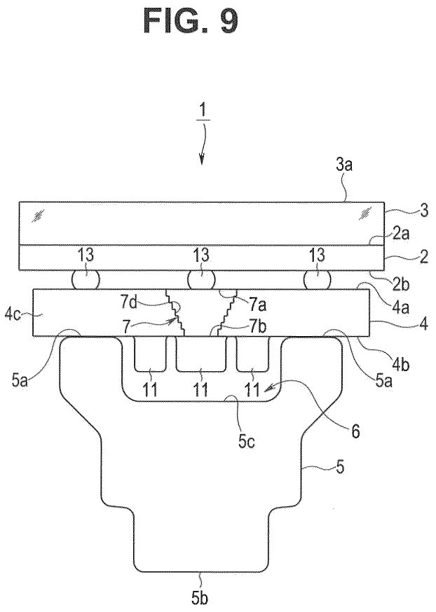

As shown in FIG. 9, both wall surfaces 7d of the cutout 7 may have a stepped shape. In other words, both side surfaces as wall portions of the cutout 7 are formed to be the stepped shape such that the dimension is gradually decreased from the opening of the distal end side (first base 7a side) to the opening of the proximal end side (second base 7b side) in a direction along the one side surface 4c of the planar circuit substrate 4. That is, the cutout 7 is formed such that the width dimension is stepwise decreased from the opening on the front surface 4a side which is the distal end surface of the planar circuit substrate 4 to the opening on the rear surface 4b side which is the proximal end surface.

Note that in the present modification, in the cutout 7, the first base 7a of the opening on the front surface 4a side of the planar circuit substrate 4 is set to be longer than the second base 7b of the opening of the rear surface 4b side of the planar circuit substrate 4 (that is, the second base 7b is shorter than the first base 7a).

### Second Modification

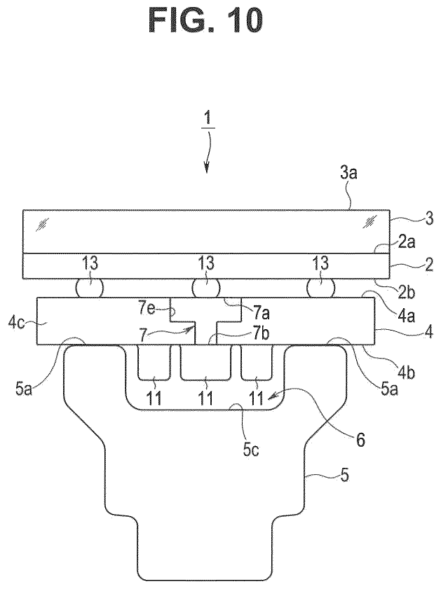

As shown in FIG. 10, both wall surfaces 7e of the cutout 7 may be formed in crank-shaped, and an opening along the one side surface 4c of the planar circuit substrate 4 may be in T-shaped. That is, in the cutout 7, the width dimension of the both side surfaces as wall portions is stepwise decreased from the opening on the front surface 4a side which is the distal end surface of the planar circuit substrate 4 to the opening on the rear surface 4b side which is the proximal end surface.

Note that in the present modification, in the cutout 7, the first base 7a of the opening on the front surface 4a side of the planar circuit substrate 4 is set to be longer than the second base 7b of the opening on the rear surface 4b side of the planar circuit substrate 4 (that is, the second base 7b is shorter than the first base 7a).

### Third Modification

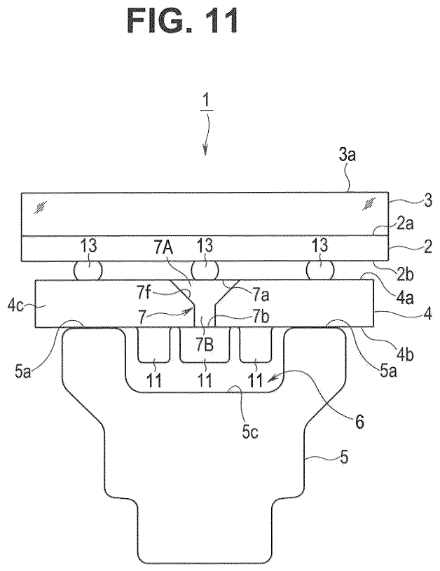

As shown in FIG. 11, the cutout 7 may be formed in a tapered shape on a distal end side of both wall surfaces 7e, and an opening along the one side surface 4c of the planar circuit substrate 4 may be in a funnel shape. That is, the cutout 7 includes a first cutout 7A formed to be a tapered shape in which a dimension is gradually decreased toward the front surface 4a side which is the distal end surface of the planar circuit substrate 4. The cutout 7 includes a second cutout 7B that is provided on the rear surface 4b side which is the proximal end surface of the planar circuit substrate 4, has an equal width dimension to the second opening, and is communicated with the first cutout 7A.

Note that in the present modification, in the cutout 7, the first base 7a of the opening on the front surface 4a side of the planar circuit substrate 4 is set to be longer than the second base 7b of the opening of the rear surface 4b side of the planar circuit substrate 4 (that is, the second base 7b is shorter than the first base 7a).

### Fourth Modification

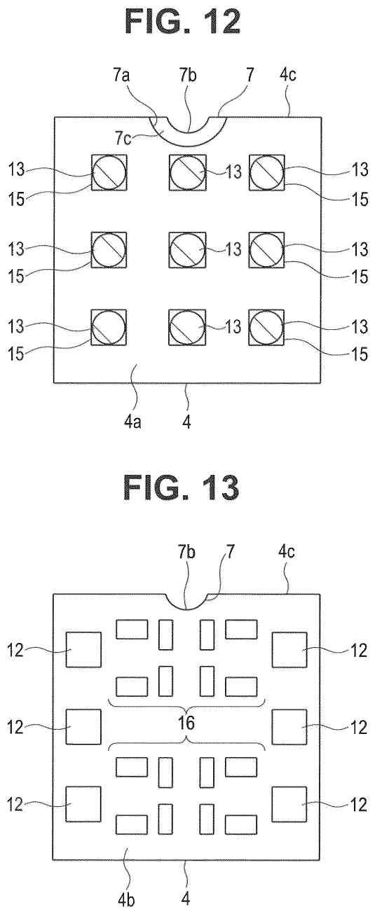

As shown in FIGS. 12 and 13, openings of the cutout 7 in the front surface 4a and the rear surface 4b of the planar circuit substrate 4 may be a semi-circular shape (or arc shape equivalent to a semi-circle). The tapered wall 7c of the cutout 7 is a shape of a curved side surface of a semi-circular cone. With such a structure, it is easier to insert the distal end of the dispense nozzle 130 in the cutout 7 when filling the underfill agent 10.

### Fifth Modified Example

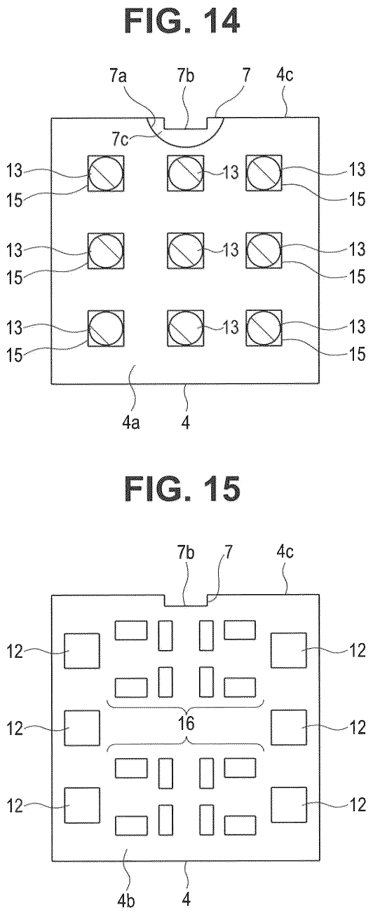

Note that as shown in FIGS. 14 and 15, the opening of the cutout 7 in the front surface 4a of the planar circuit substrate 4 may be a semi-circular shape (or arc shape equivalent to a semi-circle), and the opening in the rear surface 4b of the planar circuit substrate 4 may be rectangular.

### Sixth Modification

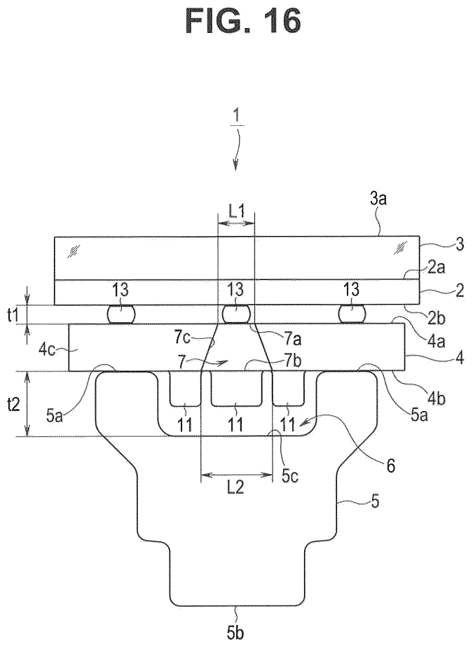

As shown in FIG. 16, in the cutout 7 formed in the one side surface 4c of the planar circuit substrate 4, the length L1 of the first base 7a on the front surface 4a side may be shorter than the length L2 of the second base 7b on the rear surface 4b side (L1<L2). That is, the cutout 7 of the present modification is formed such that the width dimension of the opening on the rear surface 4b side (second base 7b side), which is the proximal end surface, is larger than the width dimension of the opening of the front surface 4a side (first base 7a side), which is the distal end surface of the planar circuit substrate 4 in the direction along the one side surface 4c of the planar circuit substrate 4.

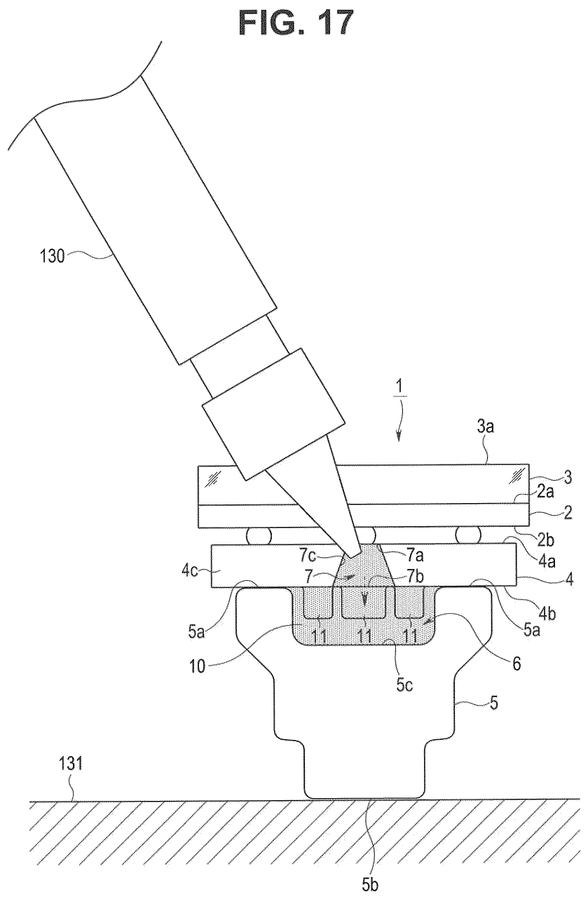

The image pickup unit 1 of the present modification as above is also applied with the underfill agent 10 from the cutout 7 in the application process of the underfill agent 10 as shown in FIG. 17.

At this time, the underfill agent 10 flows to the recessed portion 6 side of the odd-shaped circuit substrate 5 on the vertically downward side, as shown in FIG. 17. After the underfill agent 10 is filled in the recessed portion 6 of the odd-shaped circuit substrate 5, the underfill agent 10 rises vertically upwards from the cutout 7 by the
operation of the capillary action, and flows into the gap between the image pickup device 2 and the planar circuit substrate 4.

That is, the length L1 of the first base 7a of the cutout 7, which is the width dimension of the opening on the side of the gap between the image pickup device 2 and the planar circuit substrate 4, is set to be shorter than the length L2 of the second base 7b of the cutout 7, which is the width dimension of the opening on the side of the gap between the planar circuit substrate 4 and the odd-shaped circuit substrate 5 (L1>L2).

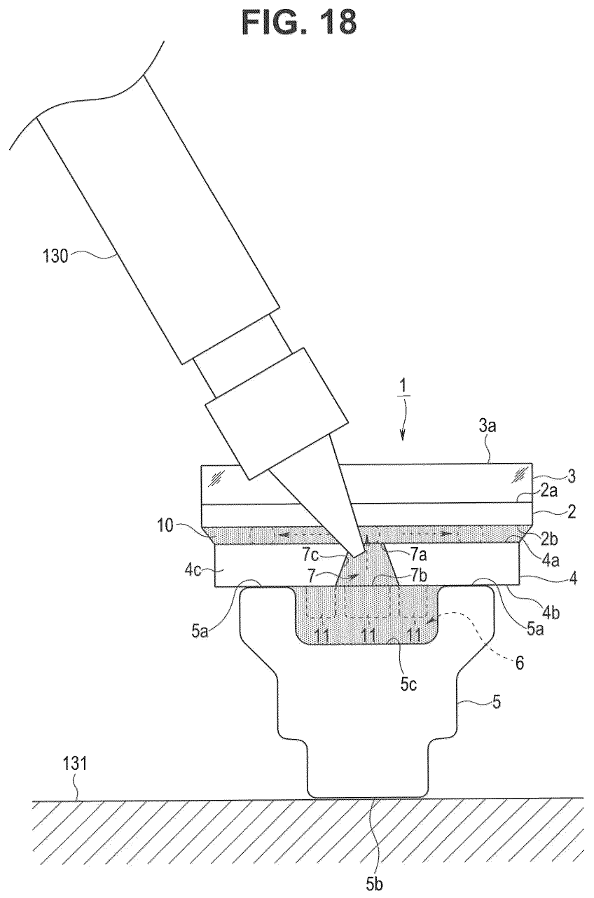

Also in the present modification, the distance t1 of the gap between the image pickup device 2 and the planar circuit substrate 4 is set to be shorter than the length L2 of the second base 7b (t1<L2). Thus, by the principle of capillary action, the underfill agent 10 flows into the gap between the image pickup device 2 and the planar circuit substrate 4 on the vertically upward side against gravity, as shown in FIG. 18.

The image pickup unil of the present modification is provided with the cutout 7 for applying the underfill agent 10 by the dispense nozzle 130 in the one side surface 4c of the planar circuit substrate 4. The cutout 7 has a structure where the opening on the odd-shaped circuit substrate 5 side is wider than the opening on the image pickup device 2 side.

Also in the present modification, in the cutout 7, the length L1 which is the width dimension of the opening on the image pickup device 2 side is set to be longer than the distance t1 of the gap between the image pickup device 2 and the planar circuit substrate 4 (L1>t1).

The cutout 7 has a penetration shape including openings on both of the image pickup device 2 side and the odd-shaped circuit substrate 5 side in the one side surface 4c of the planar circuit substrate 4. Therefore, after filling the underfill agent 10 in the recessed portion 6 of the odd-shaped circuit substrate 5, the underfill agent 10 flows to the gap between the image pickup device 2 and the planar circuit substrate 4.

With this, in the image pickup unit 1, the underfill agent 10 can be filled in the gap between the image pickup device 2 and the planar circuit substrate 4 and the gap between the planar circuit substrate 4 and the odd-shaped circuit substrate 5 (in the recessed portion 6).

With this, in the configuration of the cutout 7 of the image pickup unit 1 in the present modification, the underfill agent 10 applied from the cutout 7 can be filled while preventing from flowing outside. As a result, an external shape of the image pickup unit 1 can be prevented from becoming larger due to flowing out of the underfill agent 10.

The disclosure described in the embodiment and the modifications explained above is not limited to the embodiment and the modifications. Besides, at an implementation stage, various modifications can be implemented in a range not departing from the gist of the disclosure. Further, disclosures at various stages are included in the embodiment and the modifications explained above. Various disclosures can be extracted by appropriate combinations in a disclosed plurality of constituent elements.

For example, when the described problems can be solved and the described effects can be obtained even if several constituent elements are deleted from all the constituent elements described in the embodiment and the modifications, a configuration from which the constituent elements are deleted can be extracted as a disclosure.

What is claimed is:

1. An image pickup unit comprising:
an image pickup device, a first substrate, and a second substrate, the image pickup device, the first substrate, and the second substrate being connected in order from a distal end of the image pickup unit,
the image pickup unit further comprising:
a cutout that is provided on a side surface of the first substrate and penetrates from a distal end surface to a proximal end surface, the cutout being formed such that a width dimension of a second opening on a side of the proximal end surface is smaller than a width dimension of a first opening on a side of the distal end surface in a direction along the side surface; and
a resin filled in the cutout, a gap between the image pickup device and the first substrate, and a gap between the first substrate and the second substrate.

2. The image pickup unit according to claim 1, wherein when a distance between a rear surface of the image pickup device and the distal end surface of the first substrate is t1, and a length of the first opening in a width direction is L1, L1>t1 is set.

3. The image pickup unit according to claim 1, wherein when a distance between a rear surface of the image pickup device and the distal end surface of the first substrate is t1, and a length of the second opening in a width direction is L2, L2>t1 is set.

4. The image pickup unit according to claim 1, wherein when a length of the first opening in a width direction is L1, and a length of the second opening in a width direction is L2, L1>L2 is set.

5. The image pickup unit according to claim 1, wherein:
the second substrate includes a recessed portion that is opened on a distal end side and has a bottom surface; and
when a distance between a rear surface of the image pickup device and the distal end surface of the first substrate is t1, and a distance between the proximal end surface of the first substrate and the bottom surface of the recessed portion of the second substrate is t2, t2>t1 is set.

6. The image pickup unit according to claim 1, wherein:
the second substrate includes a recessed portion that is opened on a distal end side and has a bottom surface; and
when a distance between a rear surface of the image pickup device and the distal end surface of the first substrate is t1, a distance between the proximal end surface of the first substrate and the bottom surface of the recessed portion of the second substrate is t2, a length of the first opening in a width direction is L1, and a length of the second opening in a width direction is L2, L1>t1, L1>L2, L2>t1, and t2>t1 are set.

7. The image pickup unit according to claim 1, wherein:
the first substrate includes a plurality of connection lands on the distal end surface and the proximal end surface; and
when, in the cutout, a depth of an opening on the side of the distal end surface of the first substrate in a direction orthogonal to the side surface of the first substrate is d1, a depth of an opening on the side of the proximal end surface of the first substrate in the direction orthogonal to the side surface of the first substrate is d2, a distance between the side surface and a first connection land closest to a side of the side surface of the plurality of connection lands provided on the distal end surface is D1, and a distance between the side surface and a second connection land closest to the side of the side surface of the plurality of connection lands provided on the proximal end surface is D2, d1≤D1 and d2≤D2 are set.

8. The image pickup unit according to claim 1, wherein the cutout is formed such that dimensions of both side surfaces of the cutout are gradually decreased from the first opening to the second opening in the direction along the side surface.

9. The image pickup unit according to claim 1, wherein the cutout is formed such that width dimensions of both side surfaces of the cutout are stepwise decreased from the first opening to the second opening in the direction along the side surface.

10. The image pickup unit according to claim 1, wherein the cutout includes a first cutout that is provided on the side of the distal end surface, a dimension of the first cutout in a width direction being gradually decreased from the first opening to the side of the proximal end surface, and a second cutout that has an equal length in a width direction to the second opening and is communicated with the first cutout.

11. An endoscope comprising:
an insertion portion configured to be inserted in a subject; and
an image pickup unit that is provided in the insertion portion, and includes an image pickup device, a first substrate, and a second substrate, the image pickup device, the first substrate, and the second substrate being connected in order from a distal end of the image pickup unit, wherein
the image pickup unit further comprising:
a cutout that is provided on a side surface of the first substrate and penetrates from a distal end surface to a proximal end surface, the cutout being formed such that a width dimension of a second opening on a side of the proximal end surface is smaller than a width dimension of a first opening on a side of the distal end surface in a direction along the side surface; and
a resin filled in the cutout, a gap between the image pickup device and the first substrate, and a gap between the first substrate and the second substrate.

12. An image pickup unit comprising:
an image pickup device, a first substrate, and a second substrate, the image pickup device, the first substrate, and the second substrate being connected in order from a distal end of the image pickup unit,
the image pickup unit further comprising:
a cutout that is provided on a side surface of the first substrate and penetrates from a distal end surface to a proximal end surface, the cutout being formed such that a width dimension of a second opening on a side of the proximal end surface is larger than a width dimension of a first opening on a side of the distal end surface in a direction along the side surface; and
a resin filled in the cutout, a gap between the image pickup device and the first substrate, and a gap between the first substrate and the second substrate.
